(19) United States
(12) Patent Application Publication
(10) Pub. No.: US 2005/0096849 A1
(43) Pub. Date: May 5, 2005

(54) System and method for managing geospatially-enhanced agronomic data

(76) Inventor: Robert Joe Sorrells, East Prairie, MO (US)

Correspondence Address: FISH & RICHARDSON P.C., 5000 BANK ONE CENTER, 1717 MAIN STREET, DALLAS, TX, 75201, US

(21) Appl. No.: 10/980,411

(22) Filed: Nov. 3, 2004

Related U.S. Application Data
(60) Provisional application No. 60/517,194, filed on Nov. 4, 2003.

(51) Int. Cl. G06F 17/60; G06F 19/00; G01N 33/48; G01N 33/50

(52) U.S. Cl. 702/19; 705/1

(57) ABSTRACT

A system and method for collecting, interpreting and disseminating agronomic geospatially-enhanced data is disclosed. The system collects and aggregates agronomic data based on a commodity, such as geospatially-enhanced data, from a variety of local and remote data sources. The system layers the data to form a matrix of data for the particular commodity. Utilizing the method and system, users can access geospatially-enhanced data from the system for a variety of purposes, such as product improvement and improved market efficiencies. Users of the system and method may easily be both producers and consumers of data.

### RELATED APPLICATION

This application claims the priority under 35 U.S.C. §119 of provisional application Ser. No. 60/517,194 filed Nov. 4, 2003.

### TECHNICAL FIELD

This invention relates to geospatially-enhanced information and, more particularly, to a system and method capable of managing agronomic geospatially-enhanced data.

### BACKGROUND

For any particular commodity, there are a number of participants in one or more markets centered on or providing that commodity. For example, in a generic sense, such a market would likely include at a minimum at least one producer of the commodity and at least one consumer for the commodity. Additionally, any given consumer could also be a producer of the commodity and vice versa. Other participants could be present between the source for the commodity and the ultimate consumer for the commodity. Moreover, vendors, suppliers, consultants, service providers and others related to the commodity are likely present in the market. The more information the various participants in a market have about the particular commodity and the effects each has on the market, the more efficiently the market can operate. It will be generally understood that “commodity,” as used herein, is intended to include both traditional notions of commodities (i.e., articles of commerce such as crops), as well as more modern notions of commodities, including virtually any thing of use, profit or advantage, such as data and other intellectual property and logical entities.

Turning now to the context of agriculture, by way of example, a primary producer and consumer of geospatially-enhanced data is a farming operation, or, more specifically, a farmer. As used herein, “farmer” may reference or include any entity or individual operable to produce, plant, reap, or manage crops including corporation, organizations and associations, and others. Additional producers and/or consumers of such data would include virtually every other participant in the agriculture industry, such as manufacturers of agricultural products (e.g., fertilizers, herbicides, etc.), vendors and suppliers of agricultural products, agronomic information service providers, agriculture fulfillment operations, food processing entities, financial services providers (e.g., bankers, insurers), merchandisers/commodity brokers, local, state and federal governments and agencies (e.g., USDA), and the like.

Currently, the various producers and/or consumers associated with geospatially-enhanced data often utilize manual systems and processes in an attempt to produce and utilize such data. Examples of such processes include manual tracking of field data, such as crop yields, product applications, etc., manual production and submission of reports to governmental agencies, insurance companies, financial institutions, and manual tracking of point-of-sale (POS) information and use of delivery tickets for elevator operations. Such manual systems and processes suffer from a number of significant disadvantages, including, without limitation, lack of integration with related systems and/or processes, lack of geo-reference-based processing, and need for multiple entry of same data, resulting in slow, unintelligent, relatively complex and inefficient distribution channels within the industry.

There have been limited attempts at automating one or more components or processes within the agriculture industry. For example, systems have been announced/designed to assist a specific participant within the agriculture industry with regard to that participant's specific role in the industry. Examples of such systems include SoilTeq's “AgCentral ONLINE”™ product. Such systems suffer from specific and narrow focus. For example, the AgCentral ONLINE™ product is designed exclusively for use by agriculture product dealers (e.g., fertilizer dealer) and merely provides such dealers with a data warehousing/archiving service for a limited number of data layers (e.g., yield, soil tests, fertility recommendations, and applied data). Such systems are currently closed, are typically not web-enabled, and do not appear to provide universal and comprehensive support to the various participants within the agriculture industry.

Other systems are even more specific in focus and assistance. For example, MPower³™, a crop production database owned by ConAgra, is targeted to technical specialist farming, a very small percentage of farming operations. Additionally, the system is closed and is relatively expensive and complicated to use. At least in part due to such limitations, MPower³™ is no longer commercially available. Another example, the “VantagePoint™” product, was produced as a collaborative effort by Deere & Company, Farmland Industries, and Growmark, Inc. VantagePoint™ was an attempt to create a national information network connecting the farmer, the crop consultant and any other advisors with whom the farmer elected to share certain crop information. Since the system was closed, and primarily designed to assist the sale of certain agriculture products, it did not achieve success as a true information network. The product was eventually taken back in-house by John Deere and is currently not actively marketed.

### SUMMARY

The present invention comprises a system, software, and method of collecting, interpreting and disseminating geospatially-enhanced data. Each of the system, software, and method are generally capable of real time data collection, data aggregation regardless of native data formats, value-added interpretation of such data, and seamless dissemination of the value-added data to a variety of producers and/or consumers of geospatially-enhanced data. For example, the system and method of the present invention finds one of its unique features in its recognition of geospatially-enhanced data as a commodity. For purposes of this application, the term geospatially-enhanced data is used to refer to any data or information that has, or can be assigned, a geographical reference, such as a physical location. Also for purposes of this disclosure, the system and method of the present invention will be described in the context of the agriculture industry. This context is purely illustrative and is not intended to restrict the scope and/or application of the invention in any way. In other words, although the system and method of the present invention has potential application in a number of markets and/or industries, for purposes of this disclosure, the unique features and characteristics of the present invention are explained in the context of the agriculture industry. It is noted that the terms “geo-reference” and “geospatial” are used interchangeably herein.

The system, software, and method of the present invention are “open” in nature, allowing any and all of the participants in the market, occasionally limited to those with secure access, to access, provide, withdraw and manipulate data, and otherwise interact with the system. For example, in the context of agriculture, the system and method of the present invention allows multiple data producers and/or consumers (e.g., farmers) to input in real time geospatially-enhanced data associated with role(s) within the market (in this example, their farms and farming operations). Once in the system, such data is aggregated with other relevant data (i.e., other related geo-referenced data) both already within the system and available from remote or external resources (e.g., public and private third party databases) and otherwise interpreted to provide additional value to the data. Next, the data is made available to the various consumers and/or producers of such data, allowing such participants to more efficiently perform in the overall market.

In certain embodiments, the techniques of the present invention break down into three primary areas: (1) collection of data (both geospatially-enhanced data and raw data to which geo-reference information can be provided by the present invention); (2) aggregation and interpretation of geospatially-enhanced data; and (3) distribution of aggregated geospatially-enhanced data. For example, data is collected by the present invention in one of several ways. Primarily, data is provided to the system by the producer or manager of such data. In the context of agriculture, one such producer is the farmer. As the farmer carries out the various activities and oversees the various events associated with a modern farming operation, the farmer uses technological tools, such as bar code readers, one or more specialized or customized web sites, personal digital assistants, on-the-go yield monitors, variable rate application equipment and the like, to provide the system of the present invention with relevant data associated with his actions and the related farming operation. As merely one example of data collection, as certain products (e.g., fertilizers, insecticides) are applied to crops, the farmer records a variety of relevant information regarding such products (e.g., the manufacturer, the product, the amounts applied, etc.) into the system of the present invention. In this example, the farmer could easily and quickly capture such information via use of a bar code reader or the like, and could provide such captured information to the system via a system-linked web site associated with the farm. Another example of data capture and entry includes the use of a handheld personal digital assistant device (e.g., a Compaq® iPAQ™, preferably having GPS capabilities) to capture and wirelessly transfer to the system information regarding certain aspects of the farming operation.

Although the foregoing describes a particular example of data being provided to the system by a market participant, it is noted that many participants in a market are often both producers and consumers of data about that market. Therefore, for example, a market participant providing “raw” data to the system may also be a consumer of such data once the system has layered it with other relevant data, or otherwise added value to the raw data via interpretation, analysis, or the like. Additionally, although the example provided focuses on a farmer, the market participant could easily be any other participant within that market, such as crop consultants, agronomists, agriculture goods and services vendors and suppliers, agronomic fulfillment operations, food processing entities, financial services providers (e.g., bankers, insurers), merchandisers/commodity brokers, local, state and federal governments, and the like.

Regardless of the route of input into the system, such information is typically transferred via the system to a central repository or data store of the present system. For example, the central data store, which of course may be a distributed data store, aggregates the new data with other appropriate data in the system associated with that farm (i.e., having the same geo-reference) so that data layering can occur. Mapping software (e.g., field attribute maps) and the like can be used in connection with the aggregation features of the present invention to aggregate data down to the sub-field level. As additional data associated with a particular geospatially-referenced location is provided to the system over time, it is layered onto the existing data to provide a robust picture of all the events and activities associated with that location. Optionally, the geospatially-enhanced data within the system also can be combined with like geo-referenced data available from other sources (public and non-public), such as the National Oceanic and Atmospheric Administration (NOAA), to provide additional, complementary, or updated data (e.g., weather and climate data on the farm) and thus additional value to the information available through the present invention.

Once aggregated, the data can be interpreted in one of a variety of ways to extract additional value from the aggregation of such data. For example, querying, profiling, benchmarking and the like can be performed on the data to extract information from the data. Data mining and modeling techniques known in the relevant art can be used in conjunction with this feature of the present invention to increase the value extracted from the data within the central database of the system. Techniques employed will be dependent upon desired programming environments, size of data sets, availability of machine learning tools, and the like.

As previously mentioned, virtually every participant within a market can act as both a producer and consumer of data related to that market, having a potential interest in the value-added data made available through the present invention. Such participants include manufacturers of agricultural products vendors and suppliers of agricultural products, agronomic information service providers, agriculture fulfillment operations, food processing entities, financial services providers merchandisers/commodity brokers, local, state and federal governments, and the like. Each of these participants can utilize such data to customize, improve and better-position their products and/or services. As merely one example, once data has been entered into the system related to the planting, growth and harvesting of a particular crop (i.e., tracked from field to plate), any problems (e.g., contamination or food borne illness) attributed to the crop quickly and efficiently can be analyzed and addressed. For example, if a crop is suspected as being a potential cause of illness in consumers, the system can quickly provide information regarding the specific geographic location of the suspected source, as well as every aspect of the lifecycle of the crop to aid those evaluating the situation with information on everything from what seed type(s) were used to plant the crop, what soil type(s) were present, what climate conditions were experienced by the crop, what agriculture product(s) were used on the crop, when harvest was started/completed, what processors were provided with the crops (to contain other potential sources of illness), and the like. Once such information is available, those evaluating the crop and the claims against same are able to specifically pinpoint actions/products/sources that either confirm the claims of problems or, perhaps just as important, refute such claims, allowing the focus to be shifted towards other potential causes of the problems.

The value of the data associated with just the foregoing simple example is multi-fold. Such data not only allows for a quick and detailed location of the crop at issue (for purposes of containment, recall of products, etc.) and addressing of any concerns associated with consumption of the crop at issue, it also can: (a) guide the farmer as to what conditions (e.g., fertilizer(s), soil types, etc.) should be considered/avoided/employed for future crops; (b) educate the manufacturers of agriculture products as to bad/optimal formulations of their products with respect to various soil types and the like; (c) provide the supplier of agricultural products with data to assist with sales and inventory of same; and (d) even provide a financial institution (e.g., bank) with information to assist it in decisions regarding loans to assist the farmers, manufacturers and/or the suppliers in the market.

In one implementation of the present invention, the value-added data is available to users on a per transaction basis. In other words, one interested in the value added data merely purchases individual “transactions” (e.g., a query or a report) through the system based upon the data. In another implementation, the value added data is available via subscription. Yet another implementation provides transaction-based and/or subscription-based access to the value added data in various combinations, such as allowing a market participant to have a “basic” subscription to the system for various routine activities, but also allowing that participant to purchase, perhaps at a discount, individual transactions that are not included in the basic subscription package.

As will be evident from the disclosure provided herein, the value added data available via the present invention has value to various participants in the respective market or industry. For example, primary producers of the disparate pieces of data (e.g., farmers, crop consultants and dealers) value the data because the system collects the data, combines it with other relevant geospatially-enhanced data, and overlays all of the data so that value can be extracted from it via interpretation. In other words, the system organizes the disparate pieces of data so that information can be extracted from it. Other participants in the market hoping to do business with the crop consultant value information about the crop consultant, his or her services, and results associated with the use of such services by others in the market and with respect to specific locations, etc. Returning to the example of the agriculture industry, such data could be used by every appropriate participant in the industry to assist them in their various role(s) within that industry, including, without limitation, field management, precision farming, food and product traceability, and the like.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a block diagram of one implementation of an agronomic management system in accordance with one embodiment of the present disclosure;

FIG. 2 is a flow diagram of a product flow in accordance with one embodiment of the present disclosure illustrated in the example context of the agriculture industry;

FIG. 3 is a flow diagram illustrating example processing of agronomic data in accordance with one embodiment of the present disclosure;

FIGS. 4A-J are diagrams illustrating examples data interfaces between and uses of the example system in FIG. 1 and various categories of participants; and

FIGS. 5A-F illustrate examples of various graphical interfaces presented to particular users or participants in accordance with one embodiment of the present disclosure.

Like reference symbols in the various drawings indicate like elements.

### DETAILED DESCRIPTION

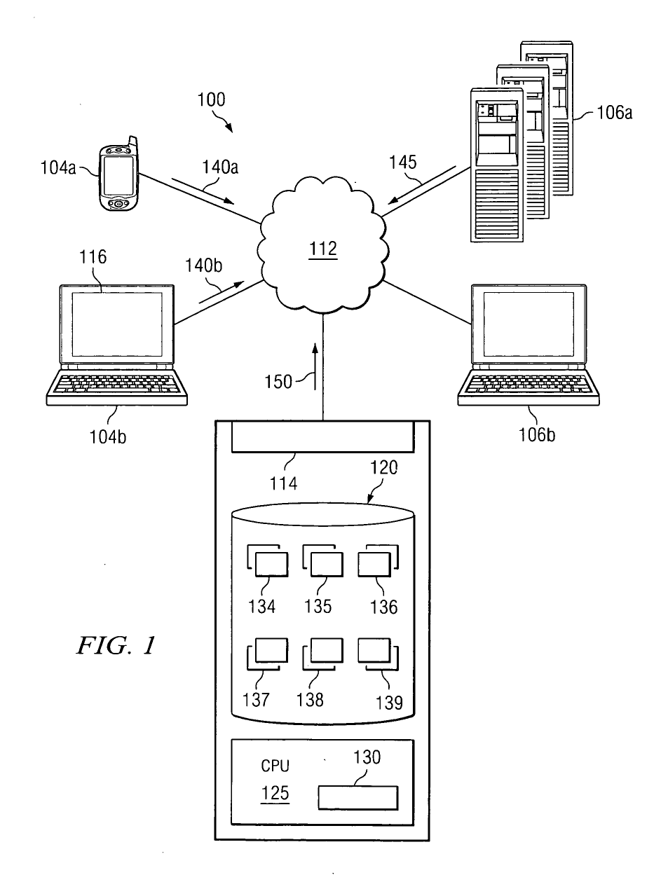

FIG. 1 illustrates an agronomic management system 100 for in accordance with one embodiment of the present disclosure. Generally, agronomic management system 100 is any system operable to receive data 140 from a plurality of data sources involved in the various stages of one or more agricultural commodities, aggregate the received data in a central data store or other repository, and to allow data analysis, reporting, and other data manipulation by various participants. Therefore, agronomic management system 100 may allow for the tracking of the life cycle of one or more agricultural products or other commodities and the participants at each stage in the cycle. Indeed, using system 100, one may be able to view certain seeds and fertilizers, as well as the manufacturers and distributors, used in the production of the selected agricultural product. Further, system 100 may provide various participants with the ability to retrieve pre-formatted information suitable for transmission to the appropriate government administration, such as the Food and Drug Administration (FDA). The data 140 provided to system 100 can be raw (i.e., in any native format) or proprietary and may be operable to include, or be capable of having assigned to it, a geo-reference, such as a geospatial tag. Put another way, data 140 generally comprises a plurality of formats, each with a number of attributes such as fields, tags, and such. For example, server 102 may receive first data 140 in a native SQL format from a first client 104a and receive second data 140 in a proprietary XML-like or XML-based format from a second client 104b. Moreover, much of data 140 is often geo-referenced or includes various geospatial attributes that allow location-oriented storage, layering, and analysis. For example, at harvest time, a particular crop may be assigned a substantially unique identifier that is spatially linked to the point of production, i.e. the farm. Also, field mapping systems and land indexing systems 106, such as the OX Spatial Index, may be used to provide or enhance data 140 coming in to the system 100 with the geo-reference or geospatial information 145 or any other geospatial attributes. In other words, while geospatial information 145 is illustrated as communicated from geospatial source 106, agronomic data 140 may include some or all of the relevant geospatial information 145.

As described below, potential users of the system and method of the present invention are nearly limitless. Examples of such users include, but are not limited to, all of the participants in a market centered on a commodity (e.g., geospatial data). Again employing the agriculture context as merely one example, potential users of the system include anyone associated with a farming operation, such as farmers, manufacturers of agriculture products, vendors and suppliers of agriculture products, food and grain processors, service providers, consultants, end users of products grown on the farm, governmental agencies and municipalities, and the like. For example, although the farmer has been used throughout this application as an example of a participant, the farmer is also a natural user of the system in that he can derive at least some value with respect to decisions made about his farm, crops, farming operations, business partners, etc., based upon information available via the system regarding his farm. The more that users and participants use the system, both as users and participants, the more complete the information available to system 100 and thus the greater the value of the data available in the system to all users.

As another example of the potential applications for this system and method of the present invention, utilizing the robust features of the system and method of the present invention, users can track and trace crops from the field to the end-user, providing new levels of safety and security for the production and consumption of food and grain products. If food is found to be contaminated, or otherwise is believed to be causing consumers to fall ill, the techniques and components provide, down to the sub-field level, data regarding every aspect of the food, its harvest location, the timing of same, the type and amount of agriculture products applied to the area(s) from which the food was harvested, the type of seed or stock from which the food was grown, the weather and climate conditions affecting the area(s) of growth, and even areas where other food has experienced the same or similar conditions (to either predict problems with foods harvested or to be harvested from an area, or to tend to rule out contamination or problems associated with the food resulting from the farming operation side of the process).

Returning to FIG. 1, agronomic management system 100 is typically a distributed client/server system that allows users of clients 104 to quickly input agronomic data 140, which includes information associated with one or more of the particular stages in the agricultural process, for use by server 102 and any of the plurality of clients 104. For example, illustrated system 100 includes server 102 that is connected, through network 112, to one or more local or remote clients 104. But system 100 may be any other suitable environment without departing from the scope of this disclosure. Moreover, it will be understood that while described in terms of agronomic data or view of the agricultural industry, this description is for illustrative purposes only and system 100 may be used to manage commodity data associated with any other suitable industry and that non-agriculture uses or implementations are within the scope of the disclosure. In short, system 100 includes at least one server 102 and a plurality of clients 104, each operable to provide data associated with the particular industry to server 102 for subsequent aggregation/normalization, dissemination (such as reporting), and other data management. The term “dynamically,” as used herein, generally means that certain processing is determined, at least in part, at run-time based on one or more variables. The term “automatically,” as used herein, generally means that the appropriate processing is substantially performed by at least part of agronomic management system 100. It should be understood that “automatically” further contemplates any suitable user or administrator interaction with system 100 without departing from the scope of this disclosure.

Server 102 includes memory 120 and processor 125 and is generally an electronic computing device operable to receive, transmit, process and store data associated with system 100. For example, server 102 may be any computer or processing device such as, for example, a blade server, general-purpose personal computer (PC), Macintosh, workstation, Unix-based computer, or any other suitable device. Generally, FIG. 1 provides merely one example of computers that may be used with system 100. For example, although FIG. 1 illustrates one server 102 that may be used, system 100 can be implemented using computers other than servers, as well as a server pool. In other words, system 100 contemplates computers other than general purpose computers as well as computers without conventional operating systems. As used in this document, the term “computer” is intended to encompass a personal or handheld computer, workstation, network computer, or any other suitable processing device. Server 102 may be adapted to execute any operating system including Linux, UNIX, Windows Server, or any other suitable operating system. According to one embodiment, server 102 may also include or be communicably coupled with a web server and/or a mail server.

Memory 120 may include any memory or database module and may take the form of volatile or non-volatile memory including, without limitation, magnetic media, optical media, random access memory (RAM), read-only memory (ROM), removable media, or any other suitable local or remote memory component. In this embodiment, illustrated memory 120 includes geospatial data table, producer data 135, bar code data 136, point-of-sale data 137, environment data 138, and crop data 139, but may also include any other appropriate data such as an audit log, security policies, and others.

Geospatial table 134 includes any parameters, variables, policies, algorithms, or rules for high-end geographic, location, or mapping services and utilities. For example, geospatial table 134 may allow or supplement other datasets by providing information used in interactive maps for display, query, and analysis. In another example, geospatial table 134 allows system 100 to provide GIS-based reports. Regardless, server 102 is operable to store geo-referenced data 145 on a persistent or run-time basis in geospatial table 134. In one embodiment, geospatial table 134 may comprise one or more tables stored in a relational database described in terms of SQL statements or scripts. In another embodiment, geospatial data 134 may store or define various data structures as text files, eXtensible Markup Language (XML) documents, Virtual Storage Access Method (VSAM) files, flat files, Btrieve files, comma-separated-value (CSV) files, internal variables, or one or more libraries. In short, geospatial table 134 may comprise one or more fields in other data tables or data sets (such as 135-139), one table or file, or a plurality of tables or files stored on one computer or across a plurality of computers in any appropriate format. Moreover, geospatial table 134 may be local or remote and can store any type of appropriate data. For example, geospatial table 134 may include, store, or reference a plurality of geospatial data layers including: i) vegetation; ii) topography; iii) cropland; iv) soils; v) yield; vi) precipitation; vii) land use; viii) land cover; ix) roads; and x) rivers.

Producer data 135, bar code data 136, point-of-sale data 137, environment data 138, and crop data 139 may each be in the same or different format, storage type, or language as geospatial data 134 such as, for example, SQL tables, XML files, open formats, and others. Moreover, each dataset may store multiple formats without departing from the scope of this disclosure. Indeed, server 102 is often operable to receive agronomic data 140 from a plurality of clients 104 in a plurality of different formats (such as layouts or languages) and normalize the received data 140 into a common or similar format or to store each in its original or cleaned format. Generally, producer data 135 includes various records or fields that help identify particular producers or farmers; bar code data 136 helps identify products used in the production of desired commodities by the producers; point-of-sale data 137 identifies various dealers, often using bar code technology; environment data 138 may include information involving soil types, climate, duration of the production cycle, and the quantity and/or identifiable quality components of a harvest (and may also specifically include or reference geospatial data 134); and crop data 139 may identify crops by a substantially unique crop ID, genetic identity of the crop, crop description, and others. Of course, the illustrated datasets are for example purposes only and memory 120 may include none, some, all, as well as other datasets without departing from the scope of this disclosure. Any or all of utilized datasets may be combined into a central repository or other data store, such as a DBMS, without departing from the scope of the disclosure.

Server 102 also includes processor 125. Processor 125 executes instructions and manipulates data to perform the operations of server 102 and may be any processing or computing component such as, for example, a central processing unit (CPU), a blade, an application specific integrated circuit (ASIC), or a field-programmable gate array (FPGA). Although FIG. 1 illustrates a single processor 125 in server 102, multiple processors 125 may be used according to particular needs and reference to processor 125 is meant to include multiple processors 125 where applicable. In the illustrated embodiment, processor 125 executes agronomic manager 130, which performs at least a portion of the aggregation and analysis of incoming agronomic data 140, correlation of the agronomic data 140 with identified or requested geospatial information 145, and allows clients 104 to view and generate reports and other outputs based on this analysis.

Agronomic manager 130 could include any hardware, software, firmware, or combination thereof operable to receive and aggregate agronomic data 140, automatically link data 140 with geospatial information 145, and provide any number of interfaces, reports, or other outputs and analyses based on the data. For example, agronomic manager 130 may be written or described in any appropriate computer language including C, C++, Java, Visual Basic, assembler, any suitable version of 4GL, and others or any combination thereof. It will be understood that while agronomic manager 130 is illustrated in FIG. 1 as a single multi-tasked module, the features and functionality performed by this engine may be performed by multiple modules such as, for example, a data inspection module, a data aggregation module, a data mining module, an imaging module, and an access module. Further, while illustrated as internal to server 102, one or more processes associated with agronomic manager 130 may be stored, referenced, or executed remotely. Moreover, agronomic manager 130 may be a child or sub-module of another software module (not illustrated) without departing from the scope of this disclosure. In one embodiment, agronomic manager 130 may include or be communicably coupled with an administrative workstation or graphical user interface (GUI).

For example, client 104 may request one of a plurality of analyses by agronomic module 130 including, for example, crop profiling, yield modeling, identity preserve tracking, and geo-referenced point-of-sale analysis. In this example, crop profiling allows users to compare attributes of particular crops to the attributes of other crops or regional attributes, thereby possibly allowing the user to identify or maximize near-premium qualities. Yield modeling allows users to substantially predict the outcome or future yield of the particular crop or field by, for example, utilizing historical data and current crop event and environmental information. Moreover, yields may be determined based, at least in part, on current real-time data such as short and long term weather and remote sensing. Identity preserve tracking provides the particular user with the ability to track certain traits of crops or commodities for domestic or international trade and may also provide or supplement geo-referenced point-of sale analysis of products and customers such as market share, market trend analysis, customer profiling, logistics distribution, inventory tracking, and restricted use pesticide tracking.

Server 102 may also include interface 114 for communicating with other computer systems, such as client 104, over network 112 in a client-server or other distributed environment. For example, server 102 often receives agronomic data 140 and/or geospatial information 145 from internal or external clients through interface 114 for storage in memory 120 and/or processing by processor 125. Generally, interface 114 comprises logic encoded in software and/or hardware in a suitable combination and operable to communicate with network 112. More specifically, interface 114 may comprise software supporting one or more communications protocols associated with communications network 112 or hardware operable to communicate physical signals.

Network 112 facilitates wireless or wireline communication between computer server 102 and any other local or remote computer, such as clients 104. Indeed, while illustrated as one network 112, network 112 may be a plurality of communicably coupled networks 112 without departing from the scope of this disclosure, so long as at least portion of network 112 may facilitate communications between clients 104 and server 102. For example, client 104 may reside in a wireless or wireline intranet that is communicably coupled to the larger network, such as the Internet. In other words, network 112 encompasses any internal or external network or networks, sub-network, or combination thereof operable to facilitate communications between various computing components in system 100. Network 112 may communicate, for example, Internet Protocol (IP) packets, Frame Relay frames, Asynchronous Transfer Mode (ATM) cells, voice, video, data, and other suitable information between network addresses. Network 112 may include one or more local area networks (LANs), radio access networks (RANs), metropolitan area networks (MANs), wide area networks (WANs), all or a portion of the global computer network known as the Internet, and/or any other communication system or systems at one or more locations.

Client 104 is any local or remote computing device operable to present the user with an interface operable to receive user commands, input, and/or queries via a GUI 116. At a high level, each client 104 includes at least GUI 116 and comprises an electronic computing device operable to receive, transmit, process and store any appropriate data associated with system 100. Client 104 may include, reference, or execute geospatial or other GPS systems, applications, or web services to supplement the input by the particular user. For example, a computer used by a distributor may include a GPS component operable to transmit, in near real time, the location of a particular product or commodity. It will be understood that there may be any number of clients 104 communicably coupled to server 102. For example, illustrated clients 104 include two remote or external clients 104, but there may be any number of internal or external clients 104. Further, “client 104,” “participant,” and “user” may be used interchangeably as appropriate without departing from the scope of this disclosure. Indeed, each user may have multiple computers or, in other cases, the computer may be used by a number of users. But, for ease of illustration, each client 104 is described in terms of being used by one user. As used in this disclosure, client 104 is intended to encompass a personal computer, touch screen terminal, workstation, network computer, kiosk, wireless data port, wireless or wireline phone, personal data assistant (PDA), one or more processors within these or other devices, or any other suitable processing device. For example, client 104 may comprise a PDA, often including global referencing capabilities (e.g., GPS), and comprising the Compaq® iPAQ™, Palm Pilots® and RIM Blackberries®, as well as offerings by Sony, Casio, Toshiba and the like. With or without GPS or other geo-referencing technology, PDAs may be used as field input devices given their relative portability (farmers can easily carry them on their person throughout the farming operations) and wireless connectivity. In other words, client 104 may comprise a computer that includes an input device, such as a keypad, touch screen, mouse, or other device that can accept information, and an output device that conveys information associated with the operation of server 102 or clients 104, including digital data, visual information, or websites via a GUI 116. Both the input device and output device may include fixed or removable storage media such as a magnetic computer disk, CD-ROM, or other suitable media to both receive input from and provide output to users of clients 104 through the display, namely GUI 116.

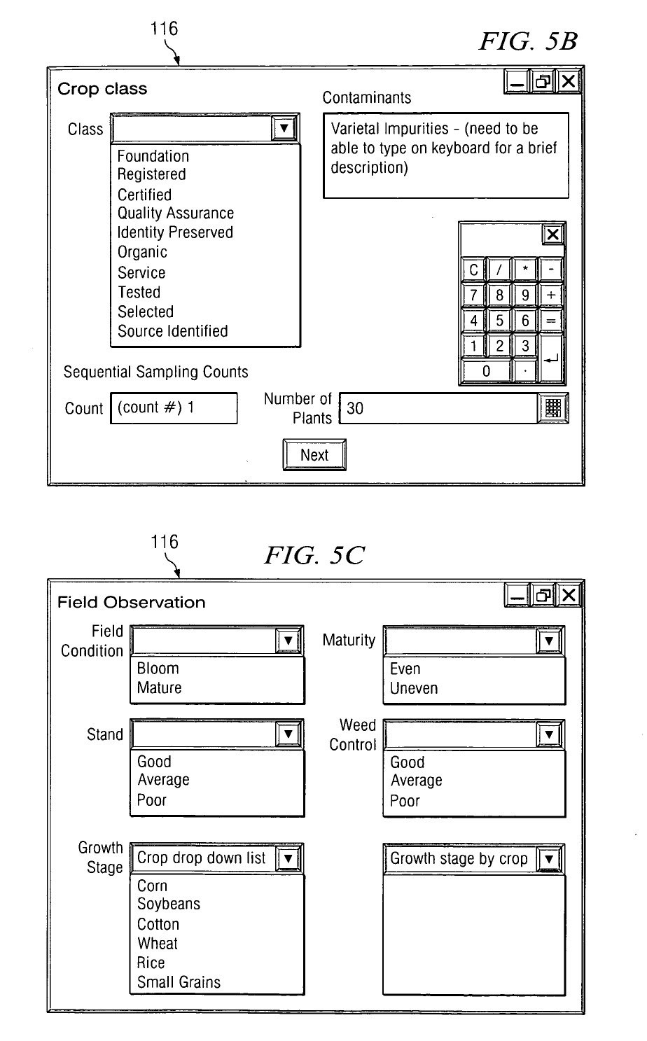

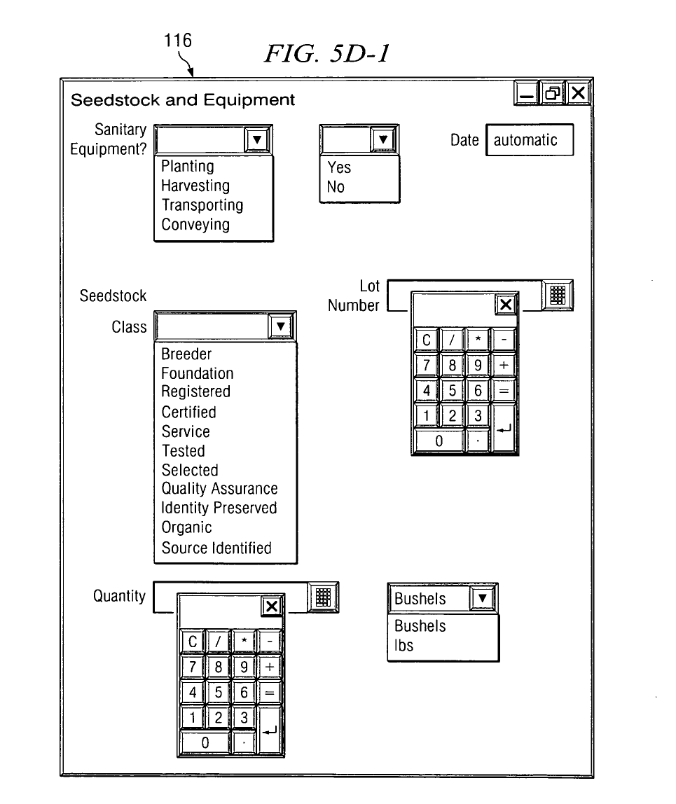

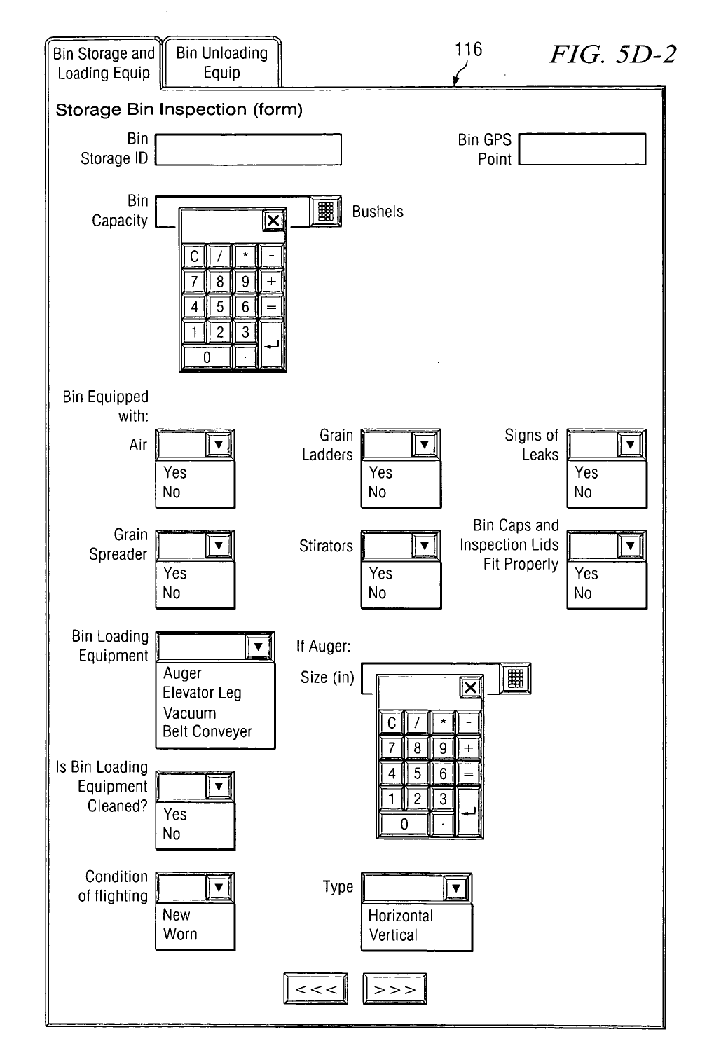

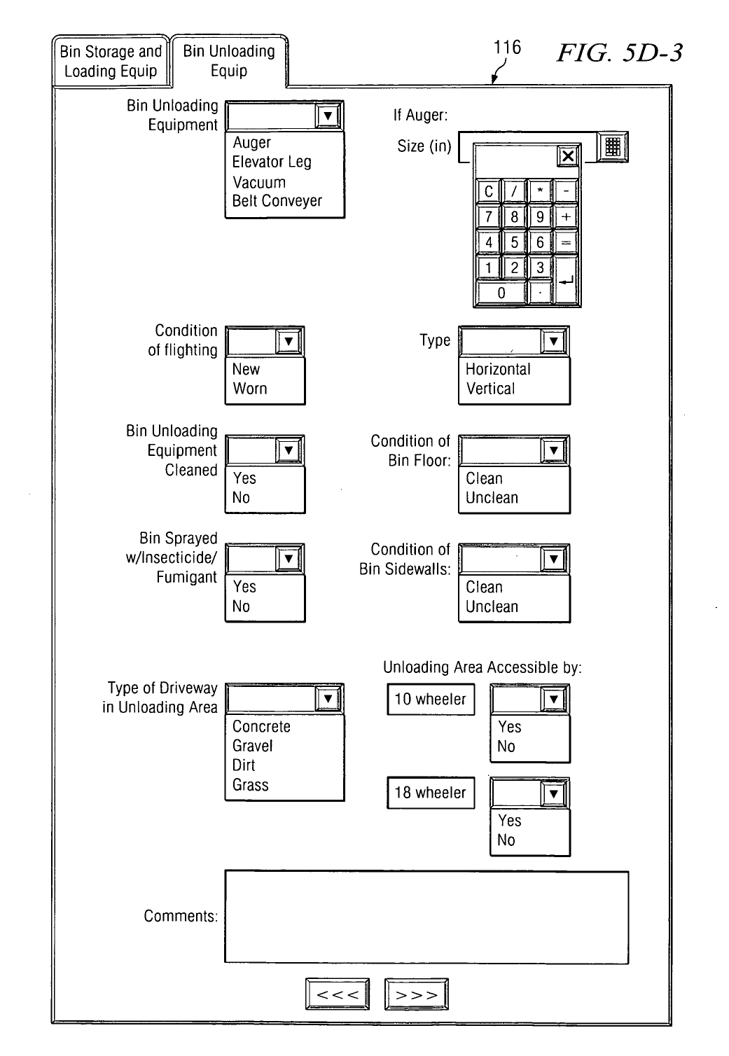

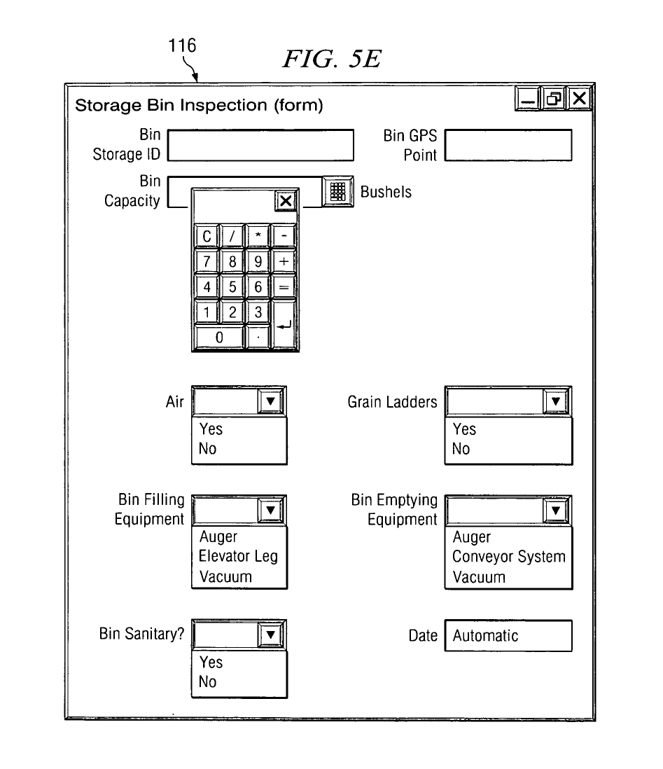

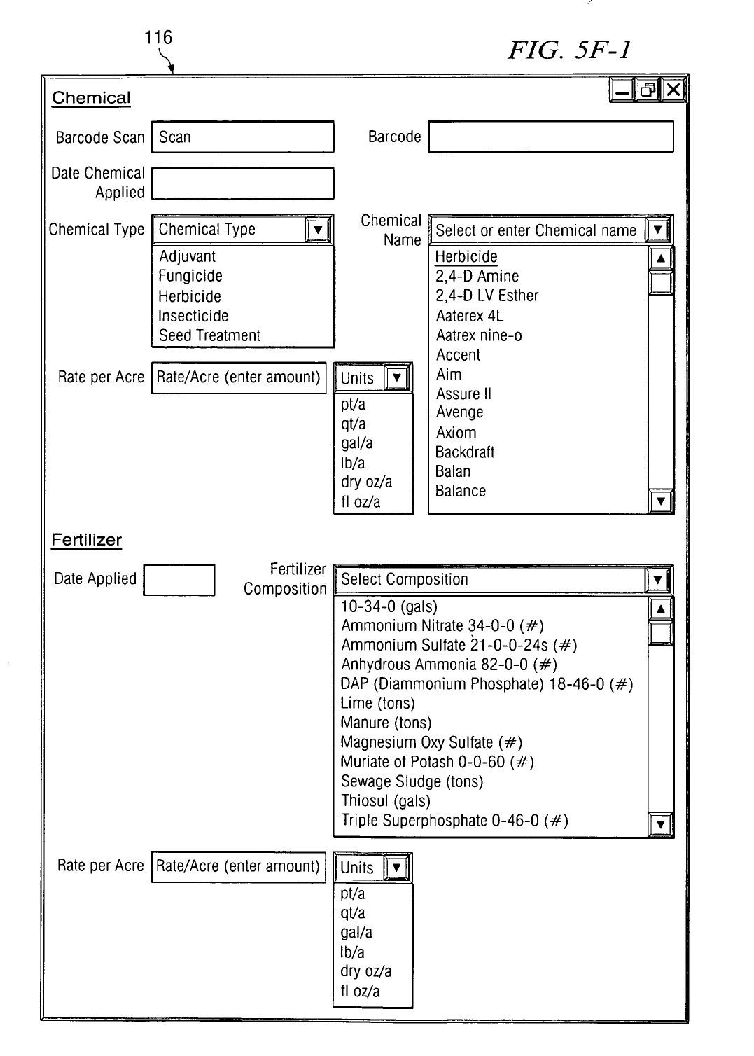

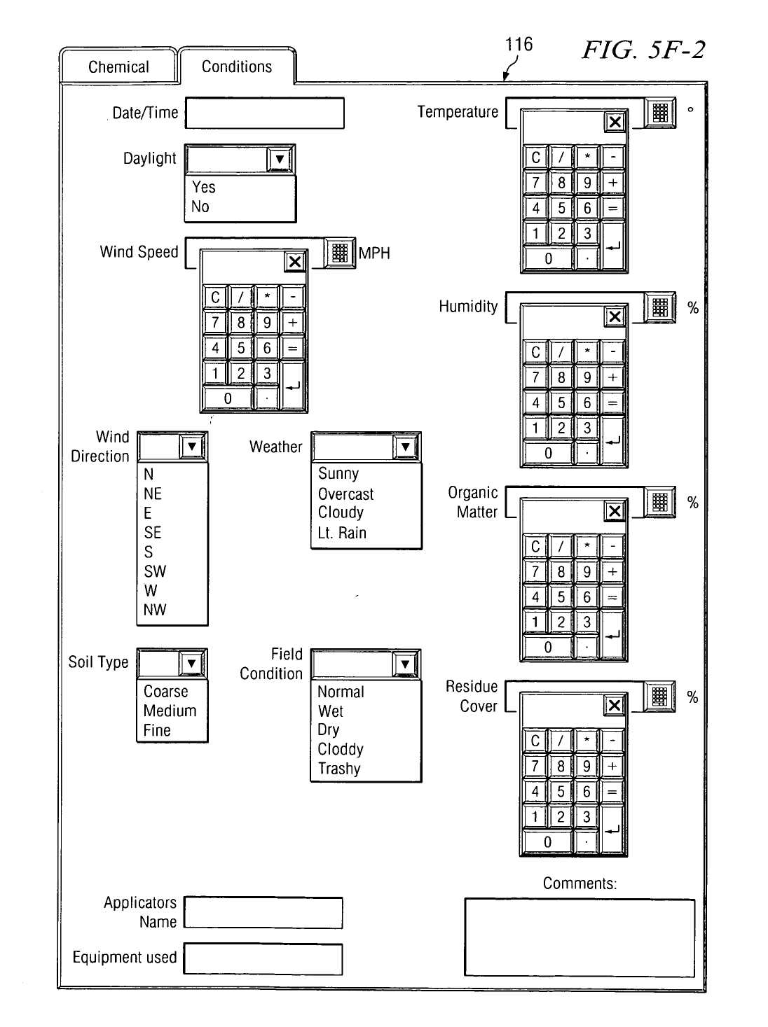

GUI 116 comprises a graphical user interface operable to allow the user of client 104 to interface with at least a portion of system 100 for any suitable purpose. Generally, GUI 116 provides the user of client 104 with an efficient and user-friendly presentation of data provided by or communicated within system 100. In certain implementations, GUI 116 presents interfaces customized to or personalized by a particular user or client 104 or based on participant status (such as producer, distributor, and such) as illustrated (for example) in FIGS. 5A-5F. In other implementations, each example GUI 116 in FIGS. 5A-5F may represent an example standard GUI that may be subsequently customized. GUI 116 may comprise a plurality of customizable frames or views having interactive fields, pull-down lists, and buttons operated by the user. Moreover, it should be understood that the term graphical user interface may be used in the singular or in the plural to describe one or more graphical user interfaces and each of the displays of a particular graphical user interface. Therefore, GUI 116 may be any graphical user interface, such as a generic web browser or touch screen, that processes information in system 100 and efficiently presents the results to the user. Server 102 can accept data from client 104 via the web browser (e.g., Microsoft Internet Explorer or Netscape Navigator) and return the appropriate HTML or XML responses using network 112.

In one aspect of operation, data 140 is provided to the system 100 via one or more of the clients 104. For example, a first client 104a may be a computer or other device connected to the server 102 via the Internet or other network 112. Of course, a portion of this network 112 may be a wireless network converted to a switch coupled with the Internet. More specifically, one or more web sites associated with a geo-referenced area may be used to provide data to system 100. In certain implementations, the type of input device or client 104 used with system 100 depends, in part, on or is otherwise associated with the type of data that is to be provided to system 100. For example, if a farmer wanted to provide data regarding crop yields to the system 100, he may utilize output from an on-the-go yield monitor. Ideally, the output from the on-the-go yield monitor interfaces directly with the system 100 via a wireless link via, for example, a global computer network 150. Alternatively, the farmer could utilize output from the on-the-go yield monitor and enter it into the system 100 via a web site associated with the system 100, preferably available via the Internet. As described above, such data 140 could be virtually any type of information concerning the farming operation, such as the products and services used in conjunction with the farming operation, the output (e.g., crops) of the farming operation, etc. As the data is received by the system 100, it is often inspected and cleansed, if required, by agronomic module 130. Depending upon the input device used by the farmer, the data provided to the system 100 may already have associated with it a geo-reference (e.g., location identifier). For example, if the farmer uses a PDA having GPS capabilities to wirelessly send data 140 about an application product for his crop to the system, such data will already have at least some geo-reference information 145 associated with it. If the farmer utilizes a web site associated with the system 100 to provide data 140, the web site may automatically associate such data 140 with the appropriate geospatial information 145. If the farmer or client 104 does not provide the data 140 with the associated geo-reference information, agronomic module 130 may retrieve or provide such information 145 such as, for example, by prompting the farmer for the location or identification number of the farming operation or by automatically associating geospatial information to the farmer using his user ID, farm ID, or any other appropriate attribute in data 140 or through the farmer's login.

Agronomic module 130 may collect, at any suitable time, geospatial information 145 from various geographic information system (GIS) modules, applications, web services, systems, servers, or other geospatial sources 106. For example, module 130 may retrieve, receive, refresh, or otherwise collect regional or global geospatial information 145 on a daily basis regardless of received data 140. In another example, module 130 may retrieve geospatial information 145 based on received data 140. In this example, agronomic module 130 may identify a particular location based on a farm identifier or user ID from agronomic data 140 and automatically reference, download, or incorporate geospatial data 145 using the identified location. It will be understood that information 145 may be in any suitable format, whether Shape (or .sh*), open format, proprietary format, or other. Moreover, it will be understood that there may be any number of geospatial sources 106 (including zero) and that geospatial sources 106 may each be any suitable computer or processing device, application, web service, or other module or component.

Once data 140 is provided to the system 100 via one or more input devices, the data is inspected and cleansed, if required, by agronomic module 130. Although data may be inspected by agronomic module 130, it will be understood that server 102 is typically operable to accept and use data 140 in a variety of native, conventional, or proprietary formats. In other words, data 140 may be inspected to determine the native format and the geo-reference information 145 associated with it. In certain embodiments, data 140 is utilized in its native format, but the data could be converted, normalized, linked, or otherwise layered, if desired, by agronomic module 130 for further processing and use by the system 100. Put another way, agronomic module 130 accepts incoming data and matches and aggregates it with other data within system 100 (i.e., data integration) having the same or similar geo-reference, bar code, product description, crop ID, and/or based on any other suitable attribute, parameter, or rule. More specifically, the data aggregation module 130 may read geo-reference information 145 associated with the incoming or stored data 140, compare it with other geo-reference information 145 associated with other data located in memory 120, and integrate (e.g., layer) the new data 140 with existing data 140 having the same geo-reference information 145 or other attribute to form a matrix of data associated by the one or more selected attributes. By way of example, and not limitation, agronomic manager 130 may access data available through the National Oceanic and Atmospheric Administration (NOAA), for example via www.noaa.org, to combine weather and climate data for a particular area of land (i.e., geo-reference identifier) and associate it with data 140 stored in memory 120 of the server 102 for that particular area of land (i.e., having like geo-reference identifier) to provide additional information regarding the land area in question and adding value to the data available via system 100.

Agronomic module 130, at any suitable time, receives and processes requests from clients 104. A user, utilizing any particular client 104, may submit a request in the form of query, request for report, or the like. In other words, such requests including queries, requests, commands, etc., and retrieves, selects, or otherwise identifies data 140 from memory 120, as well as possibly one or more outside sources 106, based on these requests. These commands may be requests for text reports, graphical elements, or formatted data pulls for governmental agencies, banks, insurance providers, or other outside entities or agencies. Of course, any client 104 may submit the request including one of the users, clients 104, or participants that submitted data 140, sources that submitted data 145, or other computers including government agencies and financial institutions. Depending upon the nature of the request, agronomic module 130 may seek additional data having the same geo-reference identifier from outside data sources, such as public and private databases, to fulfill the request. Examples of such outside data sources include climate and weather databases, land use records, governmental and municipalities records, and the like. Once system 100 has identified the data for fulfilling the request (both from the memory 120, and, if needed, from outside data sources such as geospatial sources 106), the user request is fulfilled and the user is provided with output 150. Output 150 can take any one of a number of formats, including, without limitation, a display on a screen, a printed report, an email, a faxed report, a chart, a graph and the like. Moreover, agronomic module 130 may implement various suitable techniques for processing these queries or requests or for making transmission of output 150 more efficient.

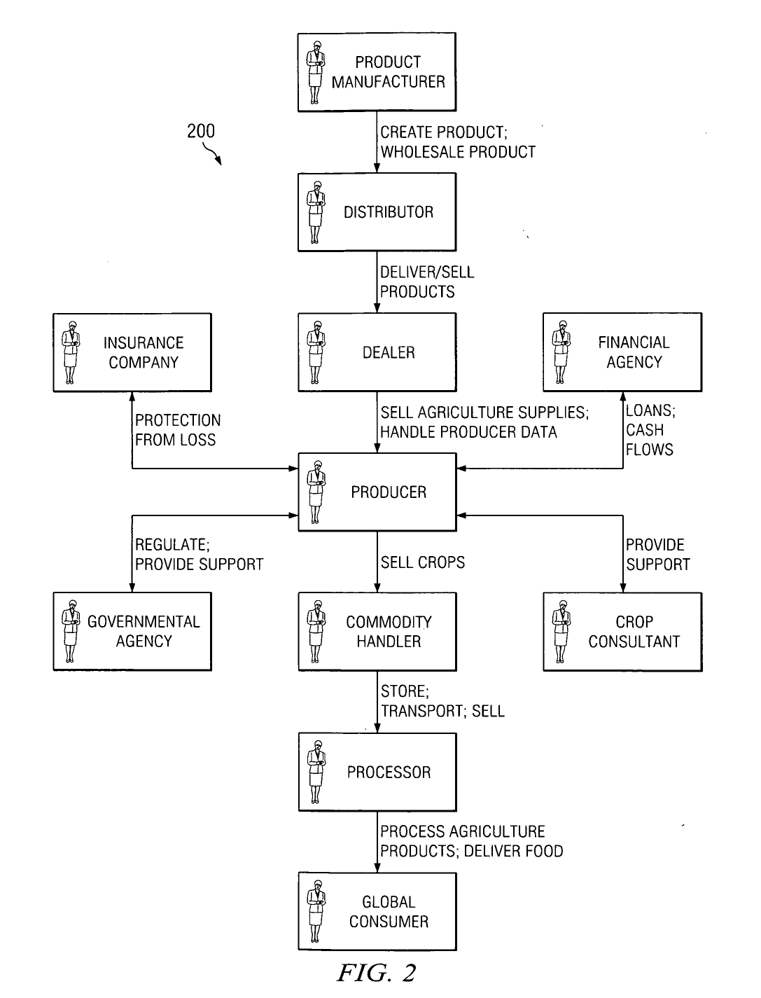

FIG. 2 is a flowchart describing an example method 200 illustrating possible uses of system 100 by various participants. At a high level, method 200 illustrates the lifecycle of a particular commodity, which system 100 is operable to track and manage. More specifically, method 200 describes the relationships among various participants or clients 104 in the agricultural supply chain, namely (for example) producers, dealers, distributors, product manufacturers, commodity handlers, processors, financial institutions, government agencies, and data analysts. But it will be understood that method 200 may include none, some, or all, as well as other, participants in any suitable industry without departing from the scope of the disclosure. Moreover, each illustrated participant may or may not implement or utilize some or all of the example techniques and actions illustrated in method 200. The following description primarily focuses on the operation of agronomic manager 130 in performing method 200, but system 100 may use any appropriate combination and arrangement of logical elements implementing some or all of the described functionality.

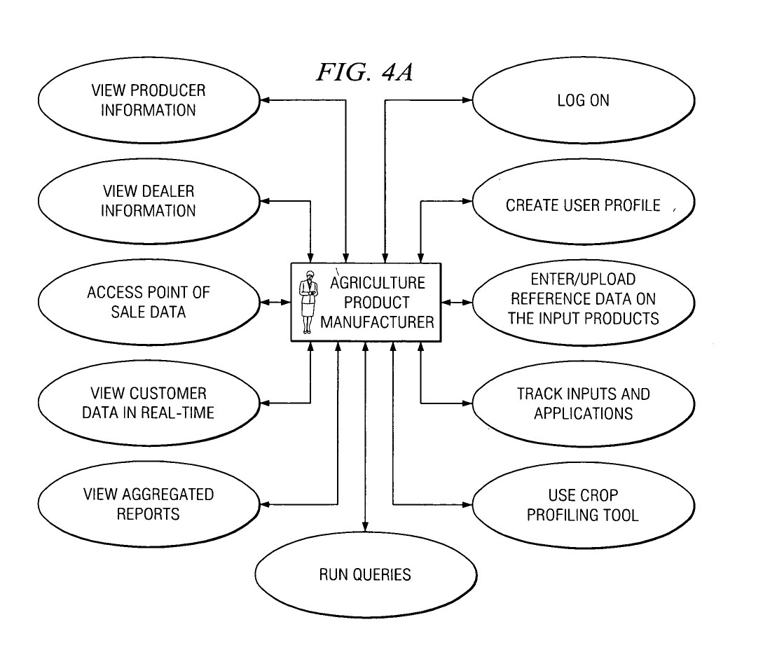

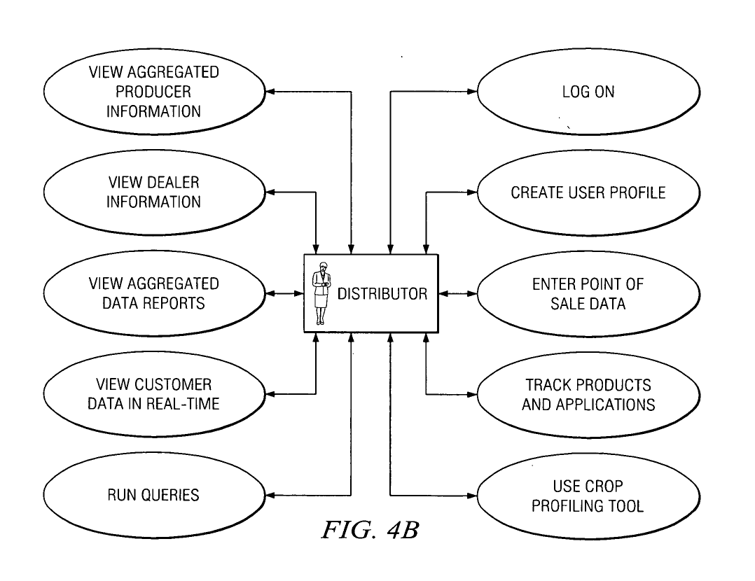

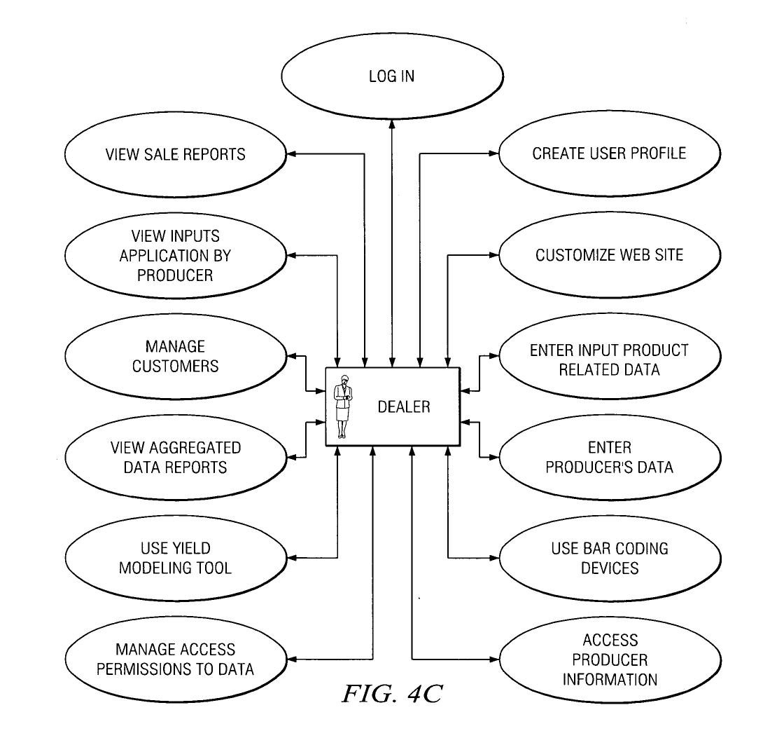

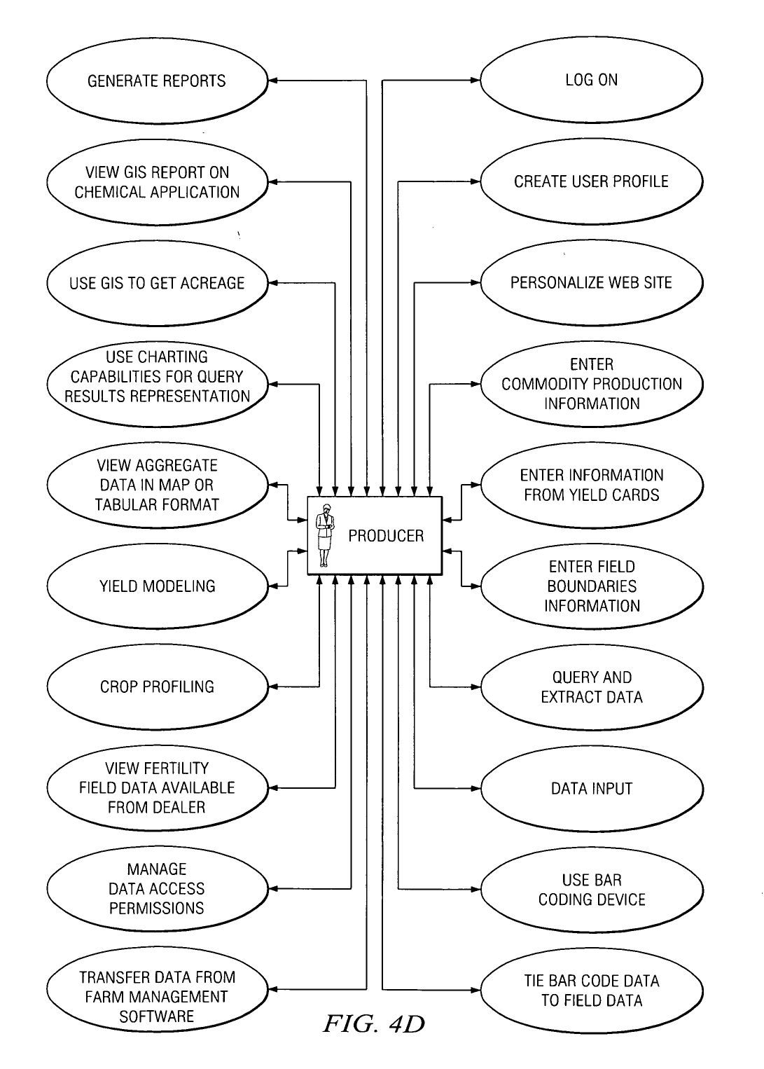

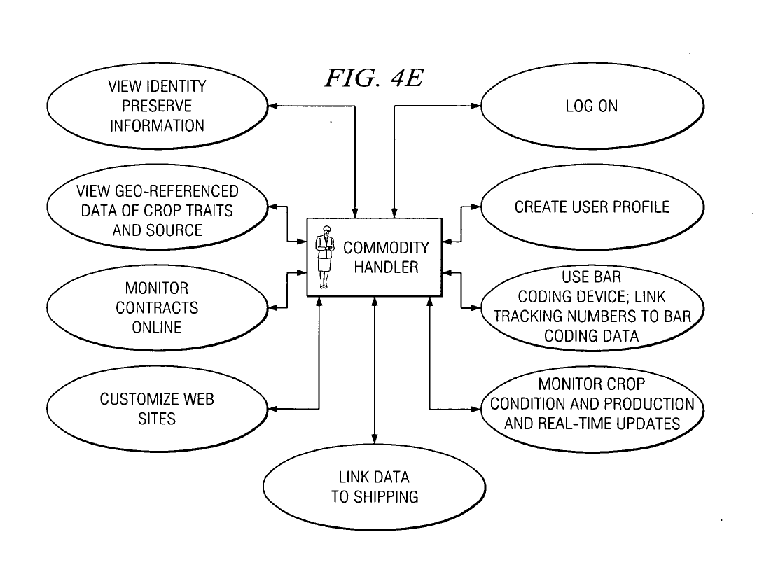

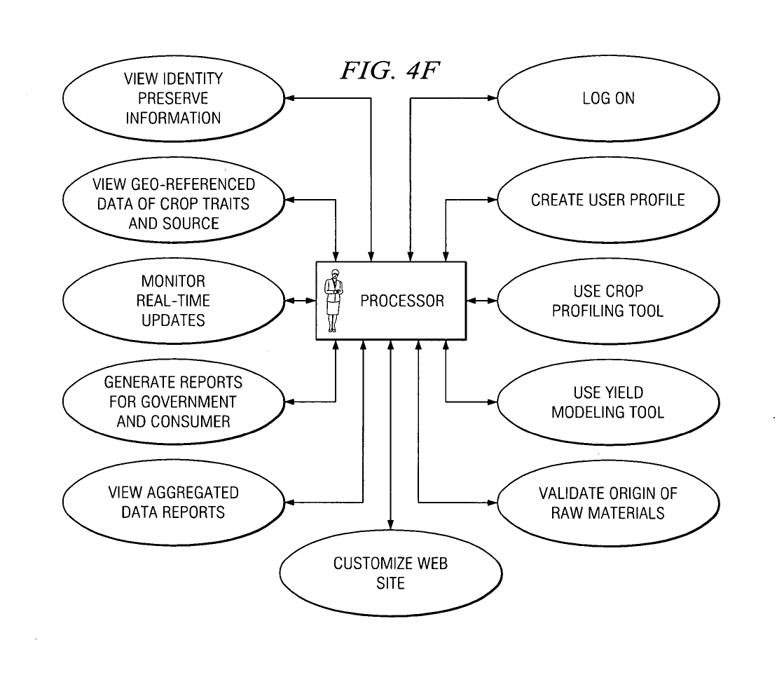

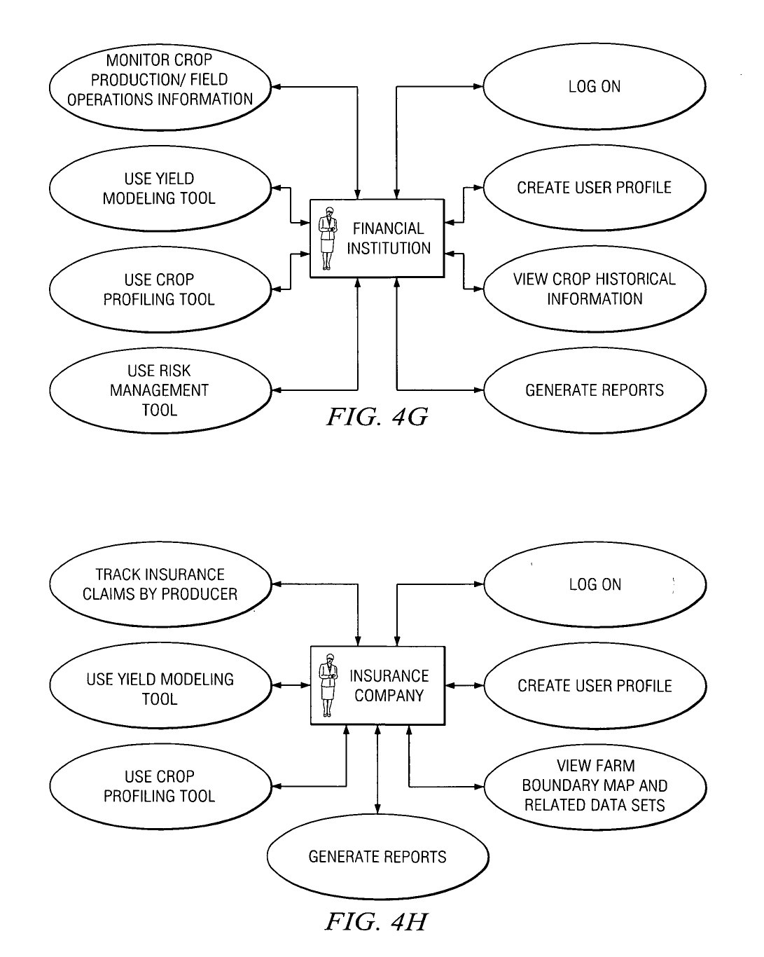

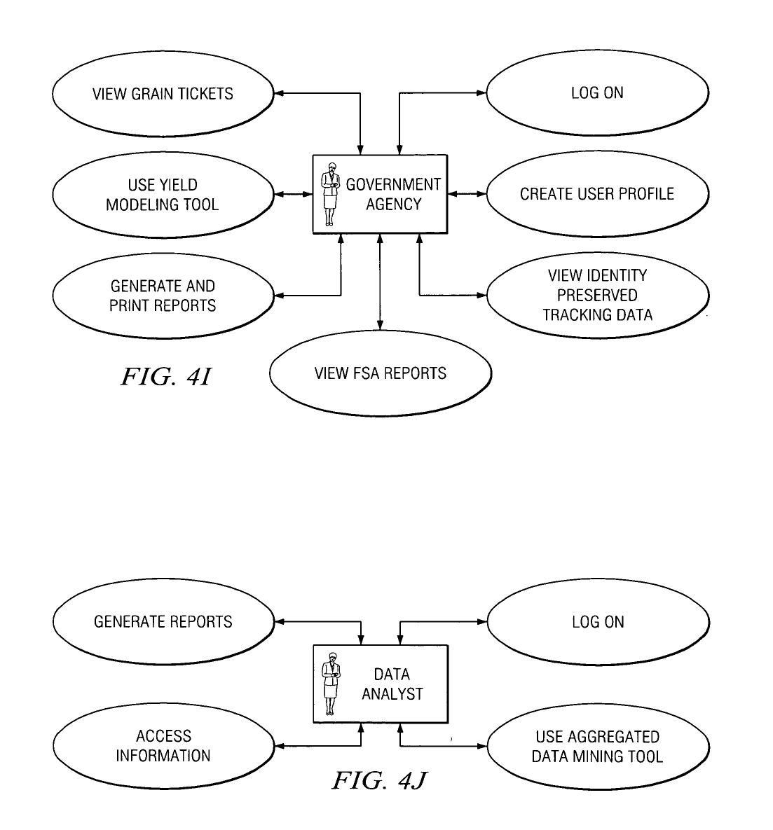

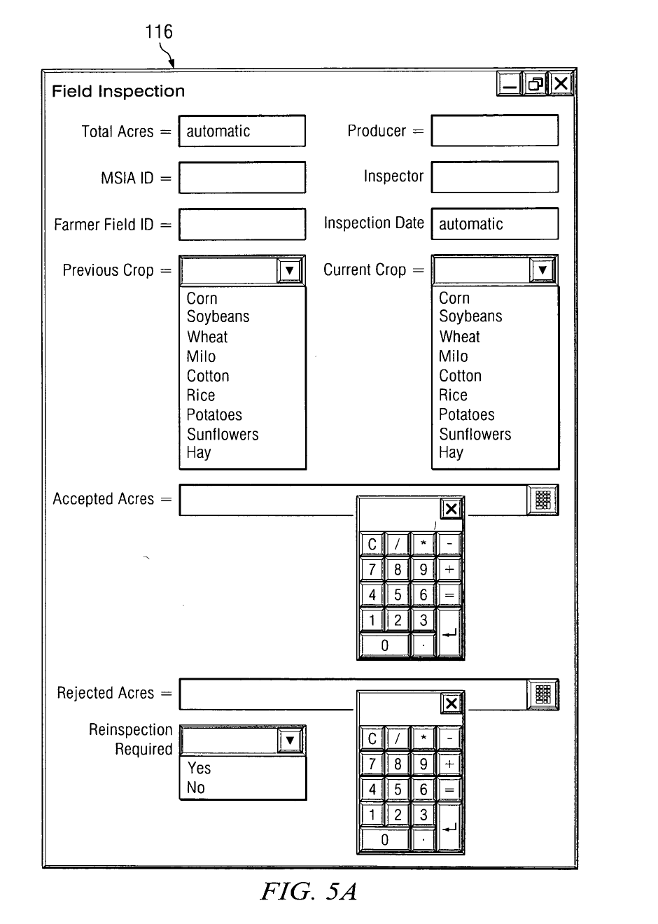

For example, the first step in the agricultural supply chain may be a product manufacturer creating or selling a product. The product manufacturer, as illustrated in FIG. 4A, may provide or upload agronomic data 140 including bar code reference data and customer point-of-sale data. Moreover, at any suitable time, the product manufacturer access agronomic module 130 to track inputs, monitor products sold and market share in real time, identify authorized and unauthorized products, and execute or request other data queries and reports, thereby possibly improving planning capabilities and customer service and/or reducing customer complaints. Next, the distributor, which is typically the “middle-man” between the manufacturer and one or more dealers, provides the transportation and warehousing of various commodities. Accordingly, the distributor may transmit point-of-sale agronomic data 140 allowing for geo-referencing or real-time monitoring of products, standardized bar coding and may access aggregated product information, dealer information, and customer data as shown in FIG. 4B.

Once delivered, the dealer sells the agricultural products, supplies, or other commodities. As illustrated in FIG. 4C, the dealer may provide agronomic data 140 including production environment data, point-of-sale information such as bar code data, GIS-based maps, and others. The dealer may also request data from agronomic module 130 allowing for tracking of products at a producer level, outlining selling regions, identifying appropriate chemicals or seeds based on different soil types and such, customer-needs forecasting, and retrieval of marketing data. Producers, i.e. farmers, generally buys the commodities or other agricultural supplies from the dealers in order to produce a particular product or crop. Producers may supply commodity product information, yield card information, field boundaries, crop profiles, pesticides or fertilizers used, and many other types of producer information. Moreover, the producer may access agronomic module 130 in order to, as illustrated in FIG. 4D for example, maintain near real-time record keeping, yield modeling, profile crops, remotely monitor or manage the farm, and execute other queries and requests. In certain embodiments, the producer may request loans from or provide information to financial institutions (illustrated in FIG. 4G) for credit risk assessment, provide updated coverage information to insurance providers or agents (illustrated in FIG. 4H), provide various reports to government agencies (illustrated in FIG. 4I), and/or provide any suitable information or access to crop consultants and other data analysts (illustrated in FIG. 4J). Of course, each of the receiving participants may also individually access agronomic module 130 to query or request agronomic, geospatial, and/or user data for various purposes. For example, the financial institution may profile crops in an effort maximize investments, monitor loans and investments, reduce fraud, and provide environmental reports. In another example, governmental agencies may access agronomic module 130 to monitor problems areas (drought, hail, etc.), generate environmental reports, generate economic or regulatory reports, and others.

Returning to FIG. 2, once the producer produces or receives notification of production of a crop from a contractor, employee, or agent, then the post-production participants, such as commodity handlers (illustrated in FIG. 4E), food processing entities (illustrated in FIG. 4F), or consumers may buy, process, or otherwise manage or monitor the agricultural product. For example, commodity handlers may access agronomic module 130 in order to track specific crops, match delivery of products to contracts, and track shipping of the particular commodity. In another example, the food processing entity may log in to agronomic module 130 validate the origin of raw materials or other commodities, provide reports to government agencies, generate or view yield modeling, and otherwise manage the product prior to and during processing.

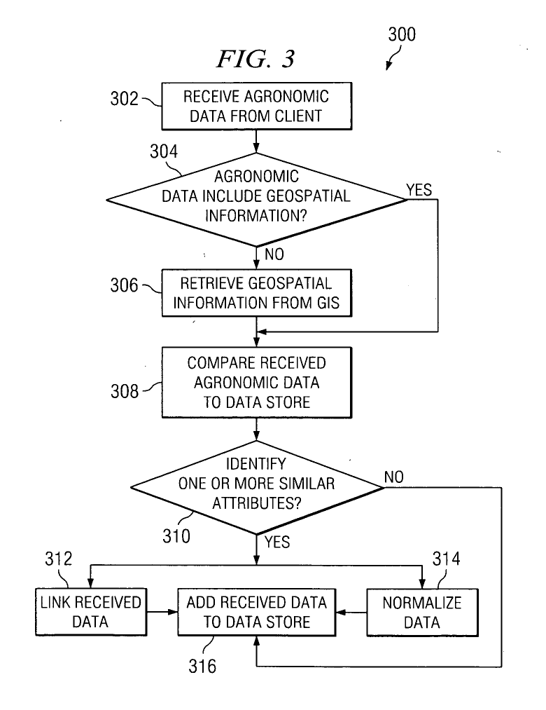

FIG. 3 illustrates method 300, which generally describes processing agronomic data 140 from a particular client 104. While describing the receipt and processing of one set of data 140 from one client 104, method 300 may be implemented or executed any number of times to process any number of data sets from any number of clients 104. Example method 300 begins at step 302, where server 102 receives a first set of agronomic data 140 from a first client 104. Next, agronomic module 130 determines whether received agronomic data 140 includes appropriate geospatial information 145 at decisional step 304. If agronomic data 140 is lacking some or all of the desired geospatial information 145, then agronomic module 130 retrieves, selects, or requests geospatial information 145 from one or more GIS entities 106 or geospatial table 134, as appropriate, at step 306. At step 308, agronomic module 130 compares the received agronomic data 140 with one or more files or tables in memory 120. For example, if received agronomic data 140 is new crop data from a farmer, then agronomic module 130 may compare agronomic data 140 to crop table 139. If agronomic module 130 identifies one or more similar attributes at decisional step 310, then agronomic module 130 may link one or more attributes of received data 140 to the particular table, normalize one or more attributes of the received data 140, or perform any other aggregation processing. Returning to the example, agronomic module 130 may identify that received data has a similar, but different, crop name for the same crop ID in crop table 139. In this example case, agronomic module 130 may then change the similar name in the received data 140 to match that in the data store. In another example case, agronomic module 130 may determine that one of the attributes in received agronomic data 140 is related to a record in another table in the data store. In this case, agronomic module 130 may link the particular data entries using foreign keys, tags, or any other suitable data component or reference. In yet another example, agronomic module 130 may cache the received data 140 until more data 140 is received and then aggregate, link, or normalize the received data 140 prior to storage in memory 120. At any point (including before, during, or after the aggregation processing), agronomic module 130 adds the received data to the appropriate table in memory 120 at step 316. It will be understood that agronomic module 130 may reformat, convert, cache, or perform other storage processes as appropriate.

Of course, the preceding steps illustrated in methods 200 and 300 are for illustration purposes only. In short, system 100 may implement, execute, or use any suitable technique for performing these and other tasks to track at least a portion of the life cycle of one or more commodities. Indeed, system 100 may track only the distribution or the crop outputs without departing from the scope of this disclosure. Accordingly, some or all of the steps in these flowcharts may take place simultaneously and/or in different orders than as shown. Moreover, system 100 may use methods with additional steps, fewer steps, and/or different steps.

Although this disclosure has been described in terms of certain embodiments and generally associated methods, alterations, and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure.

1. Software for managing agronomic data operable to:
receive first agronomic data in a first format from a first of a plurality of clients;
receive second agronomic data in a second format from a second of the plurality of clients;
store the received first and second agronomic data in a data store; and
correlate the agronomic data with geospatial information.

2. The software of claim 1, further operable to normalize the received first and second agronomic data prior to storage in the data store.

3. The software of claim 2, the first format of the first agronomic data comprising a plurality of attributes, the second format of the second agronomic data comprising a plurality of attributes, and the software operable to normalize the received first and second agronomic data prior to storage in the data store comprising software operable to convert a first attribute of the first agronomic data into a new attribute based on the data store.

4. The software of claim 2, the first format of the first agronomic data comprising a plurality of attributes, the second format of the second agronomic data comprising a plurality of attributes, and the software operable to normalize the received first and second agronomic data prior to storage in the data store comprising software operable to link a first attribute of the second agronomic data to a first attribute of the first agronomic data.

5. The software of claim 4, further operable to layer the received first agronomic data and the second agronomic data in the data store using the linked attribute.

6. The software of claim 1, further operable to retrieve the geospatial information from a remote data source.

7. The software of claim 6, the retrieved geospatial information comprising Shape files.

8. The software of claim 1, the first agronomic data comprising a plurality of attributes, at least one of the attributes comprising a geospatial attribute, and the software further operable to link the first agronomic data to the geospatial information based, at least in part, on the geospatial attribute.

9. The software of claim 1, the first client comprising an agricultural product manufacturer and the first agronomic data comprising bar codes and associated product descriptions.

10. The software of claim 9, the second client comprising an agricultural product distributor and the second agronomic data comprising point-of-sale information and at least one of the bar codes.

11. The software of claim 10, further operable to:
receive third agronomic data in a third format from a third of the plurality of clients, the third client comprising a farmer and the third agronomic data comprising crop profile, fertilizer usage, geospatial farm data, and environmental information; and
store the received third agronomic data in the data store.

12. The software of claim 11, further operable to:
receive fourth agronomic data in a fourth format from a fourth of the plurality of clients, the fourth client comprising a food processing entity; and
store the received fourth agronomic data in the data store.

13. The software of claim 1, further operable to communicate data from the data store to a governmental agency in response to a request from one of the clients.

14. The software of claim 1, further operable to generate a report illustrating the life cycle of an agricultural product based on the data store in response to a request from one of the clients.

15. A method for managing agronomic data comprising:
receiving first agronomic data in a first format from a first of a plurality of clients;
receiving second agronomic data in a second format from a second of the plurality of clients;
storing the received first and second agronomic data in a data store; and
correlating the agronomic data with geospatial information.

16. The method of claim 15, further comprising normalizing the received first and second agronomic data prior to storage in the data store.

17. The method of claim 16, the first format of the first agronomic data comprising a plurality of attributes, the second format of the second agronomic data comprising a plurality of attributes, and wherein normalizing the received first and second agronomic data prior to storage in the data store comprises converting a first attribute of the first agronomic data into a new attribute based on the data store.

18. The method of claim 16, the first format of the first agronomic data comprising a plurality of attributes, the second format of the second agronomic data comprises a plurality of attributes, and wherein normalizing the received first and second agronomic data prior to storage in the data store comprising linking a first attribute of the second agronomic data to a first attribute of the first agronomic data.

19. The method of claim 18, further comprising layering the received first agronomic data and the second agronomic data in the data store using the linked attribute.

20. The method of claim 15, further comprising retrieving the geospatial information from a remote data source.

21. The method of claim 20, the retrieved geospatial information comprising Shape files.

22. The method of 15, the first agronomic data comprising a plurality of attributes, at least one of the attributes comprising a geospatial attribute, and the method further comprising linking the first agronomic data to the geospatial information based, at least in part, on the geospatial attribute.

23. The method of claim 15, the first client comprising an agricultural product manufacturer and the first agronomic data comprising bar codes and associated product descriptions.

24. The method of claim 23, the second client comprising an agricultural product distributor and the second agronomic data comprising point-of-sale information and at least one of the bar codes.

25. The method of claim 24, further comprising:
receiving third agronomic data in a third format from a third of the plurality of clients using a personal data assistant (PDA), the third client comprising a farmer and the third agronomic data comprising crop profile, fertilizer usage, geospatial farm data, and environmental information; and
storing the received third agronomic data in the data store.

26. The method of claim 25, further comprising:
receiving fourth agronomic data in a fourth format from a fourth of the plurality of clients, the fourth client comprising a food processing entity; and
storing the received fourth agronomic data in the data store.

27. The method of claim 15, further comprising communicating data from the data store to a governmental agency in response to a request from one of the clients.

28. The method of claim 15, further comprising generating a report illustrating the life cycle of an agricultural product based on the data store in response to a request from one of the clients.

29. A system for managing agronomic data comprising:
memory storing an agronomic data store; and
one or more processors operable to:
receive first agronomic data in a first format from a first of a plurality of clients;
receive second agronomic data in a second format from a second of the plurality of clients;
store the received first and second agronomic data in the data store; and
correlate the agronomic data with geospatial information.

30. The system of claim 29, the processors further operable to normalize the received first and second agronomic data prior to storage in the data store.

31. The system of claim 30, the first format of the first agronomic data comprising a plurality of attributes, the second format of the second agronomic data comprising a plurality of attributes, and the processors operable to normalize the received first and second agronomic data prior to storage in the data store comprising the processors operable to convert a first attribute of the first agronomic data into a new attribute based on the data store.

32. The system of claim 29, the first format of the first agronomic data comprising a plurality of attributes, the second format of the second agronomic data comprising a plurality of attributes, and the processors operable to normalize the received first and second agronomic data prior to storage in the data store comprising the processors operable to link a first attribute of the second agronomic data to a first attribute of the first agronomic data.

33. The system of claim 32, the processors further operable to layer the received first agronomic data and the second agronomic data, in the data store using the linked attribute.

34. The system of claim 29, the processors further operable to retrieve the geospatial information from a remote data source.

35. The system of claim 34, the retrieved geospatial information comprising Shape files.

36. The system of 29, the first agronomic data comprising a plurality of attributes, at least one of the attributes comprising a geospatial attribute, and the processors further operable to link the first agronomic data to the geospatial information based, at least in part, on the geospatial attribute.

37. The system of claim 29, the first client comprising an agricultural product manufacturer and the first agronomic data comprising bar codes and associated product descriptions.

38. The system of claim 37, the second client comprising an agricultural product distributor and the second agronomic data comprising point-of-sale information and at least one of the bar codes.

39. The system of claim 38, the processors further operable to:
receive third agronomic data in a third format from a third of the plurality of clients using a personal data assistant (PDA), the third client comprising a farmer and the third agronomic data comprising crop profile, fertilizer usage, geospatial farm data, and environmental information; and
store the received third agronomic data in the data store.

40. The system of claim 39, the processors further operable to:
receive fourth agronomic data in a fourth format from a fourth of the plurality of clients, the fourth client comprising a food processing entity;
store the received fourth agronomic data in the data store.

41. The system of claim 29, the processors further operable to communicate data from the data store to a governmental agency in response to a request from one of the clients.

42. The system of claim 29, the processors further operable to generate a report illustrating the life cycle of an agricultural product based on the data store.

43. The system of claim 29, the processors further operable to:
retrieve at least a portion of the geospatial information from an external GIS based on at least one attribute of the received first agronomic data; and
correlate the retrieved geospatial information with the received agronomic data after it is stored in the data store.
